(19) United States
(12) Patent Application Publication
(10) Pub. No.: US 2001/0025059 A1
(43) Pub. Date: Sep. 27, 2001

(54) Composition and method for treating non-bacterial prostatitis

(76) Inventor: Floyd A. Kastke, Los Angeles, CA (US)

Correspondence Address: Robert J. Schaap, Suite 188, 21241 Ventura Boulevard, Woodland Hills, CA, 91364, US

(21) Appl. No.: 09/757,358

(22) Filed: Jan. 9, 2001

Related U.S. Application Data
(63) Continuation-in-part of application No. 60/175,286, filed on Jan. 10, 2000.

(51) Int. Cl. C09K 3/00; B01F 3/08; B01F 17/00; A61K 38/46; A61K 35/78; A01N 65/00

(52) U.S. Cl. 516/70; 424/94.65; 424/732; 424/741

(57) ABSTRACT

A composition and a method for treatment of prostate related dysfunction and, particularly, non-bacterial prostatitis and, even more particularly, non-bacterial chronic prostatitis. The composition primarily relies upon the use of a bioflavonoid and, particularly, that bioflavonoid known as quercetin. The quercetin is mixed with a proteolytic digestive enzyme protease, such as bromelin and papain, as the primary active ingredients. However, the composition may optionally and beneficially include other prostatitis affecting agents, such as zinc derived from zinc gluconate, cranberry, saw palmetto, as well as some other active and non-active ingredients.

### RELATED APPLICATION

This application is a continuation-in-part of my co-pending U.S. provisional patent application Ser. No. 60,175,286, filed Jan. 10, 2000, for “Composition for Treating Non-Bacterial Prostatitis”.

### BACKGROUND OF THE INVENTION

1. Field of the Invention

The invention primarily relates to a composition and method for the treatment of non-bacterial prostatitis and, more particularly, to a composition and method for treating non-bacterial chronic prostatitis syndromes using bioflavonoids in a treatment composition and in a treatment method.

2. Brief Description of the Related Art

Prostatitis is a name commonly used for a non-specific group of prostate related problems and is often characterized by prostatic pain, which may actually adopt the form of phantom symptomatic pain. At present, the cause for many of the forms of prostatitis is not fully known. As a result, there is frequently no known cure for non-bacterial chronic prostatitis, although there are several therapies of varying effectiveness, and usually limited effectiveness. These therapies are generally designed to address the issues of pain and discomfort as well as the other symptoms arising from this condition of prostatitis, but are not specific to any effective cure or permanent treatment therefor.

Subjects having chronic prostatitis refractory to conventional therapies often present frustration to a urologist attempting to treat that condition. For patients who do not respond to antibiotic therapy, but have negative prostatic fluid cultures, the etiology is unclear. It is recognized that a certain percentage of these patients probably have persistent bacterial infections. Indeed, it is known that a certain proportion of men with culture-negative CPPS have positive bacterial signals in their prostatic fluid.

It is also known that CPPS may represent an inflammatory disregulation of an injury response leading to up-regulation, immune cell infiltration, oxidant stress, and cellular injury. Therefore, if there is detection of persistent infection, therapy with agents that block chemokine production and oxidant stress may improve these conditions.

The existence of chronic prostatitis syndromes are therefore well known and are common disorders in men, particularly as age progresses. However, very few of the available therapies are effective for treatment of chronic prostatitis, as aforesaid. There has been some evidence that bioflavonoids in general have improved the symptoms of this disorder in cases where tried. The use of the bioflavonoids has been well tolerated in tests and does provide some slight symptomatic improvement.

There have been some tests with patients using the bioflavonoid quercetin, alone. The symptomatic response of these patients taking quercetin has been significant. However, few patients ever became completely asymptomatic. Severe urinary symptoms in particular were least likely to improve with the quercetin alone. However, pain was controlled and the urinary symptoms were only treatable with alpha-blockers or anti-cholinergics.

There is still a great deal of controversy concerning the pathophysiology and the treatment which may be appropriate therefor. At present, prolonged antibiotic therapy is common for both the bacterial and the non-bacterial prostatitis. However, where microbial therapy has shown itself to be relatively ineffective, other therapies have used alpha-blockers, anti-inflammatory agents and muscles relaxants. Nevertheless, these therapies have only met with variable success. There has been some use of dimethyl sulfoxide in a 50% solution (known as Rimso 50) as an attempt to relieve some of the symptoms of this condition. However, this composition is introduced intervesically, usually by means of a catheter and therefore not an effective home use treatment.

Beyond the therapies which attempt to elevate the condition of non-bacterial prostatitis, anti-pain medication, including various tricylic medications have been employed. Biofeedback for pain control has been also employed. Even surgery has been used as a mode of treatment, but the patient frustration with this type of treatment is relatively high.

Today, the incidence of chronic non-bacterial prostatitis appears to be increasing and frequently the available modes of treatment are relatively ineffective or have only limited effectiveness. Thus, there has been a need for some therapy to treat chronic non-bacterial prostatitis.

### OBJECTS OF THE INVENTION

It is, therefore, one of the primary objects of the present invention to provide a composition which is effective in treating the symptoms of non-bacterial prostatitis through an anti-inflammatory mechanism.

It is another object of the present invention to provide a composition for treating non-bacterial prostatitis and even chronic non-bacterial prostatitis by use of a enzyme which increases the transmural intestinal absorption of a bioflavonoid for reducing any inflammatory condition.

It is a further object of the present invention to provide a bioflavonoid containing composition which primarily relies upon the use of quercetin, along with a digestive enzyme, for increasing transmural intestinal absorptivity and, hence, the functional bioavailability thereof.

It is an additional object of the present invention to provide a composition of the type stated which relies upon the use of quercetin and a combination of digestive enzymes, including, for example, bromelain and papain.

It is a salient object of the present invention to provide a method of administering a bioflavonoid containing composition to reduce the symptomatic disablement causes by inflammation of the bladder and prostate gland.

With the above and other objects in view, my invention resides in the novel features of form and components forming part of the composition and the method of treatment therefor in the present invention.

### BRIEF SUMMARY OF THE INVENTION

The present invention primarily relies upon the use of a bioflavonoid to operate as an anti-inflammatory agent for reducing the symptomatic effects of prostatitis and, particularly, chronic prostatitis. It has been found in connection with the present invention that the most preferred bioflavonoid, which has generally produced excellent results, is that of quercetin. The quercetin appears to operate as an anti-inflammatory agent and reduces inflammation of the bladder lining and the prostate gland.

Tests have revealed that the quercetin alone does not have a high bioavailability due to the fact that transmural intestinal absorption is relatively low. It has also been found in connection with the present invention that the use of one or more selected digestive enzymes will substantially increase the bioavailability of the quercetin or similar bioflavonoid and, hence, will improve the treatment of these symptomatic affects of prostatitis. two of the most effective enzymes thus found to be effective in the present invention include bromelain and papain. Any digestive enzyme having the effects of bromelain and papain is effective, although these two enzymes have been found to be the most effective in accordance with the present invention. The combination of these enzymes, Bromelain and papain, when used together, has also been found to even more substantially increase the bioavailability of the quercetin or other bioflavonoid.

It has also been found that other components in the composition are also effective in the treatment of chronic prostatitis of the non-bacterial type. For example, cranberry has been used to reduce prostatic hyperplasia. Saw palmetto is also another prostatic hyperplasia agent. Thus, these agents find effective use in the composition of the invention. In addition to the above, there are other ingredients which are effective in this composition and include, for example, a nervine agents, such as betony, and a kidney stimulant, such as scullcap. Further, a nerve depressant, such as valerian, may also be employed. In addition, a diarrhea treatment agent, such as cohosh black, may be used.

One of the important aspects of the present invention is the fact that the various components identified above are largely herbal blends. Consequently, these ingredients lack the harsh effects which is sometimes found with various pharmaceutical type compositions. Moreover, the composition not only finds an effectiveness greater than any pharmaceutical composition which may be available, but it is also made from natural ingredients.

The composition of this invention may include, for example, titanium dioxide or similar coloring agent as well as one or more other dyes. In this case, the dyes are preferably natural or herbal dyes, to the extent available. Magnesium stearate may similarly be included in the composition. In addition, the composition may be provided with a gelling agent or it may be made available in the form of a tablet.

This invention possesses many other advantages and has other purposes which may be made more clearly apparent from a consideration of the forms in which it may be embodied. These forms are described in detail in the specification and in the accompanying examples therefor. They will now be described in detail for purposes of illustrating the general principles of the invention. However, it is to be understood that the following detailed description and any drawings therefor are not to be taken in a limiting sense.

### BRIEF DESCRIPTION OF THE DRAWING

Having thus generally described the invention in general terms, reference will now be made to the accompanying drawing in which:

FIG. 1 is a schematic illustration of several levels of components which form part of the composition of the present invention.

### DETAILED DESCRIPTION OF PREFERRED EMBODIMENTS

The present invention relates to a composition for treating non-bacterial prostatitis and, particularly, non-bacterial chronic prostatitis. The composition largely relies upon the use of a bioflavonoid in combination with one of more digestive enzymes which increase the bioavailability of the bioflavonoid.

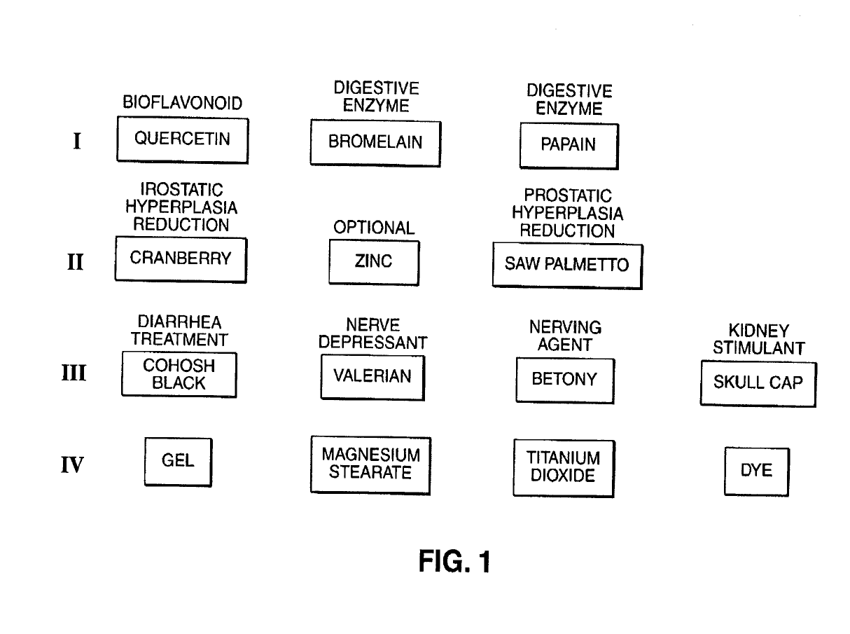

There are essentially four levels of components which are used in the composition of the present invention and which are more specifically illustrated in the attached drawing. These four levels are identified as Level I, Level II, Level III and Level IV, as shown on FIG. 1. These various levels identify those components in order of levels of importance or efficacy in the treatment of prostatitis. It has been found, in accordance with the present invention, that those components identified in Level I are the most contributing ingredients to the efficacy of the composition and with those components identified in Level II as moderately important in contributing to the efficacy of the composition. The components of Level III still contribute to the efficacy of the composition, although not necessarily with the same significance as the components of Level I and Level II. Finally, the components of Level IV are primarily designed to aid in functionality as, for example, imparting color, viscosity and the like.

Level I includes the most important of the component, as aforesaid, such as a bioflavonoid, as well as one or more digestive enzymes. These are the most critical components of the composition, since the digestive enzyme aids in the acceptance of the bioflavonoid by the body, and particularly through transmural intestinal absorption thereby increasing the functional bioavailability of the bioflavonoid. Bioflavonoids are polyphenolic compounds having anti-oxidant properties, both as free radical scavengers and as inhibitors of xanthine oxidase. The anti-inflammatory properties of these bioflavonoids block both chemokines and cytokaines. Furthermore, they possess anti-microbial properties and anti-fungal properties which may well have an impact in a treatment program using the present composition.

One of the most important bioflavonoids which has been found to be efficacious in the present invention is that known as quercetin. The quercetin acts to reduce inflammation, particularly of the bladder liner and the prostate gland, and is also used therapeutically to decrease capillary fragility. Quercetin is frequently found in eucalyptus as well as onion plants and other sources. The effects of quercetin as a common natural food ingredient is described by Albert Leung in the Encyclopedia of Common Natural Ingredients Used In Food (1980) published by John Wiley & Sons, New York, page 409 et. seq.

Quercetin can also be derived commercially from blue-green algae and is water soluble and composed of substances which often appear in fruit and vegetables as companions to vitamin C. The quercetin is primarily effective as an anti-oxidant and it is an efficacious oxidation inhibiter. It is known, for example, that linoleic and linolenic acids inhibit immune responses in many cases. Inhibition of the lipid mediator production by quercetin is believed to be, at least in part, responsible for the efficacious results in the treatment of prostatitis.

Although quercetin has been found to be one of the most effective bioflavonoids, others, which have been formally identified as vitamin P, are water soluble and include, for example, citrin, hesperindin, rutin, flavones, flavonals, and curcumin.

The bioavailability of the bioflavonoid is substantially increased with the use of digestive enzymes and, particularly, those enzymes contained in the herbal compounds, bromelain and papain. The exact mechanism for achieving the unique anti-inflammatory mechanism is not fully understood, although it is believed that these enzymes and, particularly, the bromelain and papain, are contributory to the transmural intestinal absorption which thereby gives rise to a substantially increased bioavailability of the flavonoid. Other digestive enzymes can also be used.

The bromelain is a protolytic protease enzyme which is frequently obtained from the juice of a pineapple plant. It has been used as an adjunctive therapy to reduce inflammation and edema and also to accelerate tissue repair. See, for example, M. J. T. Peatson and P. Finnegan, 1968, British Journal of Clinical Practice 22. In this respect, the bromelain actually operates, to some extent, in conjunction with the bioflavonoid to reduce inflammation.

Dietary quercetin has been found to have variable absorption depending upon the source and degree of glycosylation. It is believed that cellular bioavailability may further be compromised by binding of the quercetin to plasma proteins and, particularly, albumin. Thus, it is believed that a combination of the quercetin or other bioflavonoid, along with papain and bromelain or other dietary enzymes, will aid in transporting the bioflavonoid across the intestinal membrane.

As third level components of the composition, a prostatic hyperplasia reducing agent or combination thereof is employed. Thus, and for the purposes of this composition, it has been found that cranberry and saw palmetto are the most preferred prostatic hyperplasia reducing agents. Cranberry is know as a very useful herb for the prevention and treatment of urinary tract infections. However, cranberry is also know is lack any antiseptic or anti-bacterial properties, per se. It is known that urine of an individual becomes more acid after a large quantity of cranberry is consumed, and this acid medium does hinder bacterial development. However, it is recognized that the effectiveness of cranberry juice and cranberry generally results not necessarily from the acidifying properties, but as a result of the ability to prevent micro-organisms from adhering to the epithelial cells of the liner in the urinary tract, such as escherichia coli.

It is preferable in accordance with the present invention to use cranberry having a fructose base along with various carbohydrates and fiber and plant acids, such as benzoic acid, citric acid, malic acid and quinic acid.

Cranberry has also been found to be beneficial in reducing the effects of benign prostatic hyperplasia (prostate enlargement). The other component in the second level group of components is saw palmetto, sometimes known as sabal or serenoa. This natural herb is widely known and particularly for relieving some the urogenitle type ailments associated with prostatitis. It is known, however, that saw palmetto has some slight affect on benign prostate hyperplasia by easing urinary flow and reduced residual urine in the body, as well as increased ease in commencing micturition and decreased frequency of urination.

It is preferable to use those saw palmetto berries which are associated with tree-like palms belonging to the chinas sabal. It is believed in this case that the mechanism of increasing urinary flow and easing the commencement of micturition is anti-androgenic. It has also been found that a liposterolic extract of these berries does not induce changes in the level of testosterones in the blood plasma. It is believe that the anti-androgenic affects and the anti-inflammatory affects provided by saw palmetto account for the beneficial role of this herb, at least in the treatment of benign prostatic hyperplasia.

The saw palmetto berry is known to include a variety of fatty acids and phytosterols. In this case, the fatty acids include capric acid, caprylic acid, caproic acid, lauric acid, palmetic acid, and oleic acid, as well as the ethyl esters of these acids. It is preferable to use those saw palmetto berries which have phytosterois, such as betasitosterol, stigmasterol, cycloarterol, stigmasterol, lupeol and lupeone. It is believed that the fat soluble extract of these berries inhibit the conversion of testosterone which may responsible for the enlargement of the prostate. In addition, the saw palmetto is believed to inhibit binding of DHT to receptors in the urinary tract.

It is also possible to use other prostatic hyperplasia reducing agents, particularly of the herbal type, such as nettle root, sometimes known as urica dioica. Again, the nettle root may be used in addition to or in place of the cranberry or saw palmetto. However, as the second level components, cranberry and saw palmetto are preferred.

It is also possible to use zinc in the composition of the invention. Although the saw palmetto appears to contain a fair amount of zinc, the actual incorporation of zinc in addition to, or even in place of, the saw palmetto can also be very useful. The amount of the zinc added should be fairly small and approximately half of the amount of the saw palmetto if the latter is used. This is due to the fact that the saw palmetto itself will contain a substantial portion of zinc.

The zinc can play a vital role with regard to the immune system of the body in general and, particularly, the prostatic function. Adequate levels of zinc are desirable to insure proper function of the immune system. Moreover, zinc is desirable to obtain normal functioning of the thymus gland and the synthesis and secretion of thymic hormones. Zinc is also known to display some virus inhibiting activity. It is known that zinc concentration in the human prostate and, particularly, the normal human prostate, is very high. Thus, zinc deficiency may be a contributing factor to the higher rate of prostate enlargement. Accordingly, zinc replacement can also be an effective therapy in the composition of the invention.

The composition of the invention also includes third level components which are specifically identified in the enclosed FIG. 1 and include a diarrhea treatment agent, such as cohosh black, a nerve depressant, such as valerian, a nervine agent, such as betony, and a kidney stimulant, such as scullcap. As a result, these other Level III components are only briefly described herein. It is important to note, however, that all of the components thus far employed in the composition of the invention and, for that matter, those employed as Level III components, are natural components and are more specifically, all are herbal components. Cohosh black has been found to be suitable for a diarrhea treatment. Although it does not appear to function as a diarrhea inhibiting agent in the use of the composition of the invention, its presence has been found at least to be contributory to reduction of some of the symptomatic effects of prostatitis. Consequently, and although its exact mechanism is not known, it is frequently included in the composition of the invention. A nervine agent which is employed is betony and a nerve depressant used is valerian. Here again, these are the most preferred of the nervine agents, and nerve depressants, although other natural nervine agents and nerve depressants could be used in place thereof or, for that matter, in addition thereto. It is possible to use only valerian or for that matter betony, although each are desirable in the composition.

Scullcap or similar kidney stimulant is also included in the composition. Here again, the exact mechanism of the scullcap or other kidney stimulant is not known.

The composition of the invention also includes those Level IV components including, for example, magnesium stearate and titanium dioxide. The magnesium stearate and the titanium dioxide both operate as a binder. Various dextrins can also be used as binding agents to hold the composition in a particulate form, preferably for tableting and the like. Moreover, the composition may include a gelling agent, such as sodium hydroxymethylcellulose or the like. Natural gelling agents, to the extent available, are preferred. Finally, the composition may include one or more dyes in order to provide a color to the composition. It is preferable to use food dyes where available and, particularly, herbal dyes.

The quercetin or other bioflavonoid is used in predominant amounts in the composition and considering the quercetin and the enzymes alone as the Level I components, the quercetin can range from about 40% to about 96% by weight of the total components in the composition. The digestive enzyme bromelain would also range from about 5% to about 10% by weight of the total weight of the components used in the composition, and the papain would similarly range from about 5% to about 10% by weight of the total weight of the composition. Although smaller amounts of the bioflavonoid can be used, inasmuch as it is one of the most active ingredients in the composition, it should be present in an amount normally of about no less than 30%.

The preferred active components forming part of each dosage of the present composition is set forth in the following Table I. In this Table I, the ideal dosage percentage by weight is set forth along with a general percent weight range which can be used and also a preferred dosage percent weight range. This table does not include the inactive components, such as dyes, etc., since they would be used in amounts necessary to accomplish their intended purpose in the composition. These percent ranges are based on weight, as aforesaid.

TABLE I
Percentage of Components

| | | General | Preferred |
|---|---|---|---|
| | Ideal | Dosage | Dosage |
| | Dosage | Percentage | Percentage |
| | Percentage | Range | Range |
| Quercetin | 86.7% | 40-97% | 62-94% |
| Bromelain | 4.1% | .25-20% | 1.5-12% |
| Papain | 4.1% | .25-20% | 1.5-12% |
| Cranberry Powder | 66% | .25-25% | 2.0-18% |
| Saw Palmetto | 5.0% | .5-35% | 3-15% |
| Black Cohosh | 0.3% | 0.1-4.5% | 0.1-0.6% |
| Scullcap | 0.3% | 0.1-4.0% | 0.1-0.6% |
| Wood Betony | 0.3% | .25-20% | 0.1-0.6% |
| Valerian Root | 0.3% | 1.1-4.0% | |
| Zinc (optional) | 0.5% | .1-1.5% | .15-2.9% |

In the composition, the quercetin or other bioflavonoid is preferably used in an amount of about 500 milligrams in a total composition having a total weight of approximately 550 milligrams. However, the quercetin or other bioflavonoid can be present in widely varying amounts, as hereinafter described in more detail.

The amount of the bromelain and the amount of the papain, as well as the other ingredients, could each range as set forth in Table I above.

One preferred composition which has been tested and has effectively reduced symptoms of even chronic non-bacterial prostatitis is that set forth below in the following Table II:

TABLE II

| Components | | |
|---|---|---|
| Zinc (from | 5 | mg |
| zinc gluconate) | | |
| Quercetin | 500 | mg |
| Bromelain | 10 | mg |
| Papain | 10 | mg |
| Cranberry | 10 | mg |
| Saw Palmetto | 10 | mg |
| (berry) | | |
| Black Cohosh | 2 | mg |
| Scullcap | 2 | mg |
| Wood Betony | 2 | mg |
| Valerian Root | 2 | mg |
| Gelatin | 120 | mg |
| Magnesium Stearate | 19 | mg |
| Titanium Dioxide | <1 | mg |
| FD & C Red 40 | <1 | mg |

Another composition which is also quite effective is that described above with the addition of 5 mg of zinc. These compositions were tableted and taken in an amount of five tablets three times a day. The amount of dosage per day can vary depending on need and severity of condition.

### EXAMPLES

The invention is further illustrated by, but not limited to, the following examples:

### Example I

A composition having the following ingredients and the weight amounts per dosage set forth in the following Table III, was tested with forty subjects:

TABLE III

Weight
Amount
Dosage

| | | |
|---|---|---|
| Quercetin | 500 | mg |
| Cranberry Powder | 10 | mg |
| Bromelain | 10 | mg |
| Papain | 10 | mg |
| Saw Palmetto | 10 | mg |
| Zinc | 5 | mg |
| Black Cohosh | 2 | mg |
| Scullcap | 2 | mg |
| Wood Betony | 2 | mg |
| Valerian Root | 2 | mg |
| Gelatin | 120 | mg |
| Magnesium Stearate | 19 | mg |
| Titanium Dioxide | <1 | mg |
| FD & C Red 40 | <1 | mg |

Each of the patients consumed two to four capsules per day and continued using the capsules until the symptoms abated. It was generally found that, in most cases, the symptoms, such as urinary retention, was reduced, urination commencement was eased, and pain associated with the urinary region had been substantially reduced in about two to five days. However, it was found that in most cases when the use of the composition was stopped, there was a re-initiation of the same symptoms. Thus, it is theorized that the anti-inflammatory mechanism provided by the composition was at least in part responsible for the improved results.

### Example II

A double blind study using 30 patients without positive bacterial cultures localized to the prostatic fluid were enrolled in a double blind study. Seventeen of the patients received a composition comprised of quercetin in amount of 500 milligrams, bromelain in an amount of 10 milligrams, and papain in an amount of 10 milligrams as the active ingredients thereof. After a randomized study was completed, an additional fifteen patients were treated in an open label study with only the bromelain.

The fifteen patients which were randomized to quercetin alone completed the study and two of the fifteen patients randomized to the placebo did not because of worsening symptoms. The mean symptom score improved from 21.0 to 13.1 in the group receiving quercetin, but from 20.2 to 18.8 in the placebo group. This represented an improvement of 35%. In those patients which received only the quercetin, without the papain and bromelain, with obtainable extra prostatic secretions, the white blood cell count in the prostatic fluid decreased from 16.8 to 5.3, versus a decrease from 13.1 to 8.3 in the placebo patients. Thus, patients taking the quercetin along with the digestive enzymes showed significantly improved therapy versus those who did not.

This study proved that the patients receiving the quercetin along with at least the papain and the bromelain had superior results compared to patients which received only the quercetin.

### Example III

A composition as set forth in the following Table IV was administered to 60 male patients.

TABLE IV

Weight
Amount
Dosage

| | | |
|---|---|---|
| Quercetin | 500 | mg |
| Cranberry Powder | 10 | mg |
| Bromelain | 10 | mg |
| Papain | 10 | mg |
| Black Cohosh | 10 | mg |
| Scullcap | 10 | mg |
| Wood Betony | 10 | mg |
| Saw Palmetto | 10 | mg |
| Valerian Root | 10 | mg |
| Zinc | 5 | mg |
| Other Ingredients: | | |
| Gelatin | 120 | mg |
| Magnesium Stearate | 19 | mg |
| Titanium Dioxide | <1 | mg |
| FD & C Red 40 | <1 | mg |

The administration of the tablets again took place at a rate of two to three tablets per day until such time as the symptoms decreased on a per patient basis, much in the same manner as set forth in Example III. It was found in connection with the composition as set forth in Table IV that there was a greater mean improvement in the male patients who took the composition of Table IV, as opposed to the composition of Table III.

The composition of the invention is preferably useful as a dietary supplement. Moreover, due to the fact that the ingredients used in the composition are all natural ingredients, the otherwise deleterious effects of conventional medications used for this purpose are avoided. Although the exact theory for the pathological efficacy of the composition is not known, it is recognized that the quercetin or similar bioflavonoid changes the oxidation potential in the prostate gland. The quercetin also has been found to act as a kinase inhibitor and a nitrogen dioxide inhibitor. Hence, the stress level in prostatic fluid is effectively reduced.

The composition is not offered as a cure for either bacterial or non-bacterial chronic prostatitis. However, it is as an effective symptomatic treatment for non-bacterial prostatitis, whether or not chronic. The composition is preferably offered as a dietary supplement in capsule form. Moreover, it should be taken with meals or otherwise as directed by a physician. It should be understood that other components could also be used in the composition of the present invention. Thus, this composition is by no means limited to the specific components or the specific ingredient range as identified.

Thus there has been described and briefly illustrated a unique and novel composition for the treatment of non-bacterial prostatitis and even non-bacterial chronic prostatitis and which is based primarily on the use of natural components. The present invention thereby fulfills all of the objects and advantages which have been sought. It should be understood that many changes, modifications, variations, as well as other uses and applications will become apparent to those skilled in the art after considering this specification. Therefore, any and all such changes, modifications, variations and other uses and applications which do depart from the spirit and scope of the invention are deemed to be covered by the invention.

Having thus described the invention, what I desire to claim and secure by letters patent is:

1. A composition for the symptomatic treatment of non-bacterial prostatitis with substantially improved efficacy, said composition comprising:
a) a bioflavonoid comprised of a substantial percentage of quercetin and which exhibits anti-oxidant qualities, along with anti-inflammatory qualities; and
b) a digestive enzyme cooperative with and effective to increase the transmural intestinal absorption of the bioflavonoid and being present in an amount sufficient to achieve substantial potential absorption of the bioflavonoid in a human body in an amount substantially greater than the amount of absorption without the enzyme and in an amount effective to potentially reduce symptomatic effects of non-bacterial prostatitis.

2. The composition for the treatment of non-bacterial prostatitis of claim 1 further characterized in that said digestive enzyme is a member selected from the class consisting of bromelin and papain.

3. The composition for the treatment of non-bacterial prostatitis of claim 2 further characterized in that cranberry is present in the composition in an amount sufficient to potentially obtain prostatic hyperplasia reduction.

4. The composition for the treatment of non-bacterial prostatitis of claim 2 further characterized in that saw palmetto is present in the composition in an amount sufficient to potentially obtain prostatic hyperplasia reduction.

5. The composition for the treatment of non-bacterial prostatitis of claim 3 further characterized in that cranberry is present in the composition in an amount sufficient to potentially obtain prostatic hyperplasia reduction and saw palmetto is present in an amount sufficient to potentially obtain prostatic hyperplasia reduction.

6. The composition for the treatment of non-bacterial prostatitis of claim 1 further characterized in that said composition is provided in the form of a gel.

7. The composition for the treatment of non-bacterial prostatitis of claim 1 further characterized in that said composition is provided in the form of a tablet.

8. The composition for the treatment of non-bacterial prostatitis of claim 5 further characterized in that said composition comprises as an ingredient which can act as a nervine agent.

9. The composition for the treatment of non-bacterial prostatitis of claim 8 further characterized in that the ingredient which can act as a nervine agent is betony.

10. The composition for the treatment of non-bacterial prostatitis of claim 5 further characterized in that said composition comprises as an ingredient which can act as a kidney stimulant.

11. The composition for the treatment of non-bacterial prostatitis of claim 10 further characterized in that the ingredient which can act as a kidney stimulant is scullcap.

12. The composition for the treatment of non-bacterial prostatitis of claim 1 further characterized in that said composition comprises:
a) cranberry in an amount sufficient to potentially obtain prostatic hyperplasia reduction and in an amount of about 4% to about 9% based on the total weight of the composition;
b) saw palmetto in an amount sufficient to potentially obtain prostatic hyperplasia reduction and in an amount of about 1% to about 10% by weight based on the total weight of the composition;
c) the bioflavonoid being present in an amount of about 40% to about 97% by weight based on the total weight of the composition; and
d) digestive enzyme being present in an amount about 0.25 % to about 25% by weight based on the total weight of the composition.

13. The composition for the treatment of non-bacterial prostatitis of claim 1 further characterized in that the bioflavonoid is quercetin and a pair of digestive enzymes are used in combination.

14. The composition for the treatment of non-bacterial prostatit of claim 13 further characterized in that the digestive enzymes are bromelain and papain and each being present in an amount of about 1% to about 15% by weight based on the total weight of the composition.

15. The composition for the treatment of non-bacterial prostatitis of claim 12 further characterized in that a diarrhea treatment agent, a nerve depressant agent, a nervine agent and a kidney stimulant are also present in the composition of the invention.

16. The composition for the treatment of non-bacterial prostatitis of claim 15 further characterized in that diarrhea treatment agent is cohosh black, the nerve depressant is valerian, the nervine agent is betony, and the kidney stimulant is scullcap.

17. The composition for the treatment of non-bacterial prostatitis of claim 16 further characterized in that each of the betony, skull cap, cohosh black and valerian are present in an amount of about 0.1% to about 4% by weight by weight based on the total weight of the composition.

18. A composition for the symptomatic treatment of non-bacterial prostatitis with substantially improved efficacy, said composition comprising:
a) a bioflavonoid comprised of a substantial percentage of quercetin and which exhibits anti-oxidant qualities, along with anti-inflammatory qualities; and
b) a digestive enzyme primarily based upon bromelain and which is cooperative with and effective to increase the transmural intestinal absorption of the bioflavonoid and being present in an amount substantially sufficient to achieve substantial potential absorption of the bioflavonoid in a human body, and at least in an amount substantially greater than the amount of absorption without the enzyme and in an amount to potentially reduce symptomatic affects of non-bacterial prostatitis; and
c) a digestive enzyme, primarily based on the use of papain, operative in conjunction with the bromelain and cooperative with and effective to also increase the transmural intestinal absorption of the bioflavonoid and being present in an amount substantially sufficient to achieve substantial potential absorption of the bioflavonoid in a human body, and at least in an amount substantially greater than the amount of absorption without each of the enzymes to thereby potentially reduce symptomatic affects of non-bacterial prostatitis.

19. A process for treating non-bacterial prostatitis comprising administering to a subject having non-bacterial prostatitis a composition comprising:
a) a bioflavonoid comprised of a substantial percentage of quercetin and which exhibits anti-oxidant qualities, along with anti-inflammatory qualities; and
b) a digestive enzyme cooperative with and effective to increase the transmural intestinal absorption of the bioflavonoid and being present in an amount sufficient to achieve substantial potential absorption of the bioflavonoid in a human body substantially greater than the amount of absorption without the enzyme and in an amount effective to potentially reduce symptomatic effects of non-bacterial prostatitis.
